(19) United States
(12) Patent Application Publication
(10) Pub. No.: US 2021/0198185 A1
(43) Pub. Date: Jul. 1, 2021

(54) PROCESS FOR RECOVERING ADIPONITRILE

(71) Applicant: Ascend Performance Materials Operations LLC, Houston, TX (US)

(72) Inventors: Sanjay Dube, Madison, AL (US); Benjamin Haseltine, Houston, TX (US); Jefferson Thomas Ebert, League City, TX (US); Darrick Elmore, Pensacola, FL (US)

(21) Appl. No.: 17/138,578

(22) Filed: Dec. 30, 2020

Related U.S. Application Data
(60) Provisional application No. 62/955,075, filed on Dec. 30, 2019.

(51) Int. Cl. C07C 253/34; C07C 209/48; B01D 3/06; B01D 3/42

(52) U.S. Cl. CPC C07C 253/34; C07C 209/48; C07C 211/12; B01D 3/42; B01D 3/06

(57) ABSTRACT

A process for producing an intermediate adiponitrile stream, the process comprising separating an adiponitrile process stream comprising less than 50 wt % adiponitrile, and optionally TCH, to form the intermediate adiponitrile stream comprising at least 5 wt % adiponitrile and a heavies stream comprising high-boiling components and optionally solid impurities; and optionally utilizing at least a portion of the intermediate adiponitrile stream outside of the process.

### REFERENCE TO RELATED APPLICATION

This application claims priority to U.S. Provisional Application No. 62/955,075, filed on Dec. 30, 2019, which is incorporated herein by reference.

### FIELD

The present disclosure relates generally to recovery of adiponitrile from process streams of adiponitrile and/or tricyanohexane production processes. More specifically, the disclosure relates to the recovery of adiponitrile from a separation scheme that produces purified tricyanohexane from adiponitrile process streams.

### BACKGROUND

Cyanocarbons, e.g., organic compounds having cyano functional groups, are known and are widely used in various applications. Many of these compounds, including acrylonitrile and adiponitrile (ADN), are used as monomers to prepare various polymers, such as nylon, polyacrylonitrile, or acrylonitrile butadiene styrene. Adiponitrile, in particular, can be hydrogenated to 1,6-diaminohexane (hexamethylenediamine (HMD)) for the production of nylon-6,6. Several processes for producing cyanocarbons are known in the art. For example, one conventional adiponitrile production path utilizes the electrohydrodimerization of acrylonitrile, as described in U.S. Pat. No. 3,844,911.

This and other production methods often yield streams comprising small amounts of desirable co-products. For example, some of the conventional streams of adiponitrile production processes may contain small but not insignificant amounts of residual adiponitrile. Typically, separation of these streams has been inefficient and has not been able to effectively capture these amounts of adiponitrile. As a result, the streams are treated as waste streams, e.g., burned, which results in an outright loss of these co-products. Accordingly, valuable adiponitrile goes uncaptured.

Some ADN separation/purification processes are known. However, these processes generally relate to purification of a crude adiponitrile product stream, which comprise higher concentrations of adiponitrile.

For example, U.S. Pat. No. 3,451,900 relates to a method for the production of pure adiponitrile from a reaction product containing adiponitrile, cyclopentanone, 2-cyan-cyclopenten-(1)-yl-amine and other components higher boiling than adiponitrile wherein cyclopentanone and 2-cyan-cyclopenten-(1)-yl-amine are distilled from the adiponitrile, the improvement which comprises subjecting the reaction product to a distillation for separation into a distillate comprising adiponitrile and all lower boiling components and a residue comprising components higher boiling than adiponitrile, and thereafter submitting said distillate to a multistage vacuum distillation process for separating the lower boiling impurities from the adiponitrile.

Also, U.S. Pat. No. 6,599,398 relates to a process for the recovery of a purified adiponitrile from a mixture of adiponitrile, aminocapronitrile and hexamethylenediamine, utilizing two sequential distillations: (1) a first distillation in which the mixture is distilled in a distillation column at a head pressure that causes at least 7% of the ADN to go into the distillate, along with bishexamethylenetriamine (BHMT) and 2-cyanocyclopentylideneimine (CPI), and (2) a second distillation in which the distillate from the first distillation is distilled in a second distillation column at a head pressure sufficient to cause minimum-temperature azeotropy between adiponitrile and BHMT, thereby allowing the majority of the BHMT and CPI to be removed from the second distillation as distillate, and adiponitrile, substantially free of both BHMT and CPI, to be removed as bottoms.

Even in view of the known technology, the need exists for processes that can effectively recover amounts of residual adiponitrile from lower adiponitrile-content cyanocarbon production process streams, which results in significant improvements in overall production efficiency.

### SUMMARY

In some embodiments, the present disclosure relates to a process for producing an intermediate adiponitrile stream, the process comprising: separating an adiponitrile process stream comprising less than 50 wt % adiponitrile, and optionally TCH, to form the intermediate adiponitrile stream comprising at least 5 wt % adiponitrile and a heavies stream comprising high-boiling components and optionally solid impurities; and optionally utilizing at least a portion of the intermediate adiponitrile stream outside of the process. The separating may comprise: flashing the adiponitrile process stream to form a first intermediate adiponitrile stream comprising at least 5 wt % adiponitrile and at least 50 wt % TCH and the heavies stream and/or separating the adiponitrile process stream in one or more columns to form a second intermediate adiponitrile stream comprising at least 10 wt % adiponitrile and at least 25 wt % TCH, a heavies stream comprising high-boiling components, and a TCH stream comprising TCH and less than 10 wt. % impurities. The process may further comprise the step of purifying the intermediate adiponitrile stream, optionally via one or more distillation columns, to form a purified adiponitrile stream comprising greater than 50 wt % adiponitrile and the purified adiponitrile stream may comprise greater than 95 wt % adiponitrile and the TCH stream comprises greater than 95 wt % TCH. The first intermediate adiponitrile stream may comprise less adiponitrile than the second intermediate adiponitrile stream. The utilizing may comprise: utilizing adiponitrile in the intermediate adiponitrile stream to form hexamethylene diamine and/or combining the adiponitrile in the intermediate adiponitrile stream form an electrolyte solution. The TCH stream may comprise: TCH, from 0 wt. % to 0.05 wt. % adiponitrile, from 0 wt. % to 0.1 wt. % di(2-cyanoethyl) amine, from 0 wt. % to 0.05 wt. % cyanovaleramide, and from 0 wt. % to 0.05 wt. % tri(2-cyanoethyl) amine. In some cases, the separating of the adiponitrile process stream comprises: flashing the adiponitrile process stream to form a first intermediate adiponitrile stream comprising at least 5 wt % adiponitrile and at least 50 wt % TCH and the heavies stream, and separating the first intermediate adiponitrile stream in one or more columns to form a second intermediate adiponitrile stream comprising at least 10 wt % adiponitrile, a heavies stream comprising high-boiling components, and a TCH stream comprising at least 25 wt % TCH and less than 10 wt. % impurities. Residence time in the separating step may be less than 8 hours and/or the residence time of the intermediate adiponitrile stream in a column of the separating step at temperatures above 230° C. is less than 8 hours and/or the residence time of the intermediate adiponitrile stream in a column of the separating step at pressures above 50 torr is less than 8 hours.

### BRIEF DESCRIPTION OF THE DRAWINGS

The disclosure is described in detail below with reference to the appended drawings, wherein like numerals designate similar parts.

FIG. 1 depicts a schematic overview of an embodiment of the process for producing an intermediate adiponitrile stream.

FIG. 2 depicts a schematic overview of another embodiment of the process for producing an intermediate adiponitrile stream.

FIG. 3 depicts a schematic overview of another embodiment of the process for producing an intermediate adiponitrile stream.

FIG. 4 depicts a schematic overview of another embodiment of the process for producing an intermediate adiponitrile stream.

FIG. 5 depicts a schematic overview of another embodiment of the process for producing an intermediate adiponitrile stream.

### DETAILED DESCRIPTION

As noted above, many conventional cyanocarbon production process steams contain (lower) amounts of desirable co-products, such as adiponitrile and TCH. In conventional processes, the separation and/or recovery of these amounts of adiponitrile and/or TCH has proven to be ineffective and impractical. As a result of these separation inefficiencies, these process streams are typically vented or flared, and the desirable co-products go uncaptured.

The inventors have now found that certain separation processes provide for the effective recovery of the lower amounts of adiponitrile (and/or TCH). Because of the effectiveness of the recovery schemes, the adiponitrile is advantageously captured and may be used elsewhere or sold, which results in significant improvements in overall production efficiency. For example, the recovered adiponitrile may be conveyed (directly or indirectly) to a polyamide production process and used to make hexamethylenediamine (HMD). Importantly, when a lower adiponitrile-content streams are treated as disclosed herein, e.g., using separation units operating at low residence times and/or at low pressure, effective separation is achieved. In some cases, the particular treatment of the streams significantly concentrates the adiponitrile, which makes recovery and/or re-use practical and feasible.

Traditional purification schemes have focused on higher adiponitrile content streams, e.g., crude adiponitrile product streams. These purification schemes have proven to be ineffective and impractical for use with lower adiponitrile content streams. Because of the higher adiponitrile content (and other impurities), these schemes provide little or no guidance with regard to the aforementioned lower adiponitrile content streams. In particular, many of the higher adiponitrile content streams may comprise significant amounts of TCH and other low boiling point components.

In some cases, the present disclosure relates to processes for forming an adiponitrile stream (intermediate and/or purified). The processes comprise the step of separating a (low adiponitrile content) adiponitrile process stream to form an intermediate adiponitrile stream. The adiponitrile process stream comprises less than 50 wt % adiponitrile, e.g., the adiponitrile process stream is a low adiponitrile content, as compared to traditional crude adiponitrile product streams. The adiponitrile process stream may further comprise TCH (additional compositional information of the adiponitrile process stream is provided below). The intermediate adiponitrile stream comprises an increased amount of adiponitrile, based on the adiponitrile process stream, e.g., at least 5 wt % adiponitrile. A co-product (heavies) stream comprising high-boiling components and solid impurities is also formed from the separation of the adiponitrile process stream.

Importantly, at least a portion of the intermediate adiponitrile stream and/or the purified adiponitrile stream may, in some cases, be utilized outside of the process. As one example, the intermediate adiponitrile stream and/or the purified adiponitrile stream may be utilized to form HMD. The inventors have found that by conducting the separation in this manner surprisingly provides for a sufficiently concentrated adiponitrile stream that may be used outside of the process, e.g., for sale or in subsequent production processes. Importantly, the adiponitrile that is separated and recovered is captured and is not vented or flared as is done conventionally. Additional compositional information for the aforementioned streams is provided below. In some cases, at least 5% more residual adiponitrile is captured, e.g., at least 10%, at least 20%, at least 25%, at least 50%, or at least 75%, as compared to conventional processes, which do not treat adiponitrile process streams to recover residual adiponitrile. In some embodiments the process recovers an additional 1-5 million pounds of adiponitrile per year.

In some embodiments, the processes further comprise the step of purifying the intermediate adiponitrile stream to form the purified adiponitrile stream comprising greater than 50 wt % adiponitrile. This step, which is made increasingly effective with the initial separation step, beneficially provides for improved integration with other processes, e.g., hydrogenation of the adiponitrile to HMD.

The separations of the disclosed processes are effective and take into consideration other co-products, e.g., TCH, which can also be separated and recovered. The traditional schemes have not been found to be effective to capture both adiponitrile and TCH.

The separating step may vary, but will typically lead to the aforementioned intermediate adiponitrile stream. In some cases, the separating of the adiponitrile process stream comprises flashing the adiponitrile process stream to form a first intermediate adiponitrile stream comprising at least 5 wt % adiponitrile and at least 50 wt % TCH, and the heavies stream.

In some cases, the separating of the adiponitrile process stream comprises separating the adiponitrile process stream in one or more columns, e.g., distillation columns, to form a second intermediate adiponitrile stream. The second intermediate adiponitrile stream may comprise adiponitrile and, in some cases, at least 25 wt % TCH. The separation may further yield a heavies stream comprising high-boiling components and a TCH stream comprising TCH and (less than 10 wt. %) impurities. Compositions of the aforementioned streams are discussed in more detail below. In some cases, the first intermediate adiponitrile stream comprises less adiponitrile than the second intermediate adiponitrile stream on an overall weight basis, e.g., at least 1% less, at least 3%, at least 5%, at least 10%, at least 20%, or at least 50%.

### Adiponitrile Process Stream

As noted above, the adiponitrile process stream has a specific composition, which has surprisingly been found to separate efficiently when employing the disclosed processes. In particular, the adiponitrile process stream may comprise adiponitrile, TCH, high-boiling components, and low boiling components. Conventional separation processes have had difficulty in isolating the lower quantities of adiponitrile and/or TCH. In some embodiments, the adiponitrile process stream may be one or more process streams of another industrial chemical production process. For example, the feed stream may comprise one or more process streams from different processes or systems, e.g., the production of adiponitrile, acrylonitrile, allyl cyanide, butyronitrile, polyacrylonitrile, polyamides, polyaramids, or combinations thereof. In a specific case, the adiponitrile process stream may be one or more process streams, purge streams, or flash tails from an adiponitrile production process. In some cases, streams from multiple processes may be combined to form the stream. In conventional process, such adiponitrile-containing (and/or TCH-containing) streams are often treated as waste streams, e.g., vented or burned, and the valuable components are not recovered. By recovering adiponitrile and/or TCH from these streams, as described herein, the (residual) adiponitrile may be recovered and used or sold, thus increasing efficiency and profitability.

The adiponitrile process stream may comprise less than 40 wt % adiponitrile, e.g., less than 35 wt %, less than 30 wt %, less than wt 20%, less than 18 wt %, less than 15 wt %, less than 12 wt %, less than 10 wt %, or less than 5 wt %. In terms of ranges, the adiponitrile process stream may comprise from 0.1 wt % to 40 wt % adiponitrile, e.g., from 0.5 wt % to 30 wt %, from 1 wt % to 20 wt %, from 1 wt % to 18 wt %, from 1 wt % to 10 wt %, from 2 wt % to 15 wt %, from 3 wt % to 15 wt %, or from 5 wt % to 15 wt %. In terms of lower limits, the adiponitrile process stream may comprise greater than 0.1 wt % adiponitrile, e.g., greater than 0.3 wt %, greater than 0.5 wt %, greater than 0.7 wt %, greater than 1.0 wt %, greater than 1.5 wt %, greater than 2 wt %, or greater than 5 wt %.

In some embodiments, the adiponitrile process stream comprises less than 25 wt % TCH, e.g., less than 20 wt %, less than 18 wt %, less than 15 wt %, less than 12 wt %, less than 10 wt %, or less than 5 wt %. In terms of ranges, the adiponitrile process stream may comprise from 0.1 wt % to 25 wt % TCH, from 0.5 wt % to 23 wt %, from 0.5 wt % to 20 wt %, from 1 wt % to 15 wt %, from 1.5 wt % to 12 wt %, or from 2 wt % to 11 wt %. In terms of lower limits, the adiponitrile process stream may comprise greater than 0.1 wt % TCH, e.g., greater than 0.3 wt %, greater than 0.5 wt %, greater than 0.7 wt %, greater than 1.0 wt %, greater than 1.5 wt %, greater than 2 wt %, or greater than 5 wt %.

In some embodiments, the adiponitrile process stream comprises higher amounts of TCH. In one embodiment, the adiponitrile process stream comprises TCH in an amount ranging from 0 wt. % to 90 wt. %, based on the total weight of the feed stream, e.g., from 0 wt. %, to 89 wt. %, from 0 wt. % to 88 wt. %, from 0 wt. % to 85 wt. %, from 0 wt. % to 84 wt. %, from 10 wt. % to 90 wt. %, from 10 wt. %, to 89 wt. %, from 10 wt. % to 88 wt. %, from 10 wt. % to 85 wt. %, from 10 wt. % to 84 wt. %, from 20 wt. % to 90 wt. %, from 20 wt. %, to 89 wt. %, from 20 wt. % to 88 wt. %, from 20 wt. % to 85 wt. %, from 20 wt. % to 84 wt. %, from 30 wt. % to 90 wt. %, from 30 wt. %, to 89 wt. %, from 30 wt. % to 88 wt. %, from 30 wt. % to 85 wt. %, from 30 wt. % to 84 wt. %, from 40 wt. % to 90 wt. %, from 40 wt. %, to 89 wt. %, from 40 wt. % to 88 wt. %, from 40 wt. % to 85 wt. %, from 40 wt. % to 84 wt. %, from 50 wt. % to 90 wt. %, from 50 wt. %, to 89 wt. %, from 50 wt. % to 88 wt. %, from 50 wt. % to 85 wt. %, from 70 wt % to 90 wt %, or from 50 wt. % to 84 wt. %. In terms of upper limits, the adiponitrile process stream may comprise less than 90 wt. % TCH, e.g., 89 wt. %., less than 88 wt. %, less than 85 wt. %, or less than 84 wt. %, In terms of lower limits, the adiponitrile process stream may comprise greater than 0 wt. % TCH, e.g., greater than 10 wt. %, greater than 20 wt. %, greater than 30 wt. %, greater than 40 wt. %, greater than 50 wt %, or greater than 60 wt %, or greater than 70 wt %.

In some cases, the adiponitrile process stream also comprises low-boiling components. Generally, the low-boiling components are impurities having relatively low boiling points. For example, each of the low-boiling components may have a boiling point of less than 415° C., e.g., less than 410° C., less than 400° C., less than 395° C., or less than 390° C. Examples of low-boiling components that may be present in the adiponitrile process stream include various cyanocarbons, e.g., acrylonitrile, propionitrile, hydroxypropionitrile, monocyanoethyl propylamine, succinonitrile, methylglutaronitrile, adiponitrile, 2-cyanocyclopentylidenimine, bis-2-cyanoethyl ether, di(2-cyanoethyl) amine, di-2-cyanoethyl propylamine, cyanovaleramide and combinations thereof. In some cases, the term “lights” refers to components that have lower boiling points, e.g., lower boiling points than adiponitrile or lower boiling points than TCH.

In one embodiment, the adiponitrile process stream comprises low-boiling components in an amount ranging from 0 wt. % to 70 wt. %, e.g., from 0 wt. %, to 65 wt. %, from 0 wt. % to 60 wt. %, from 0 wt. % to 55 wt. %, from 0 wt. % to 50 wt. %, from 5 wt. % to 70 wt. %, from 5 wt. %, to 65 wt. %, from 5 wt. % to 60 wt. %, from 5 wt. % to 55 wt. %, from 5 wt. % to 50 wt. %, from 10 wt. % to 70 wt. %, from 10 wt. %, to 65 wt. %, from 10 wt. % to 60 wt. %, from 10 wt. % to 55 wt. %, from 10 wt. % to 50 wt. %, from 12 wt. % to 70 wt. %, from 12 wt. %, to 65 wt. %, from 12 wt. % to 60 wt. %, from 12 wt. % to 55 wt. %, from 1 wt % to 20 wt %, from 2 wt % to 15 wt %, from 3 wt % to 15 wt %, from 1 wt % to 10 wt %, from 12 wt. % to 50 wt. %, from 15 wt. % to 70 wt. %, from 15 wt. %, to 65 wt. %, from 15 wt. % to 60 wt. %, from 15 wt. % to 55 wt. %, or from 15 wt. % to 50 wt. %. In terms of upper limits, the adiponitrile process stream may comprise less than 70 wt. % low-boiling components, e.g., less than 65 wt. %, less than 60 wt. %, less than 55 wt. %, less than 50 wt. %, less than 20 wt %, less than 15 wt %, or less than 15 wt %. In terms of lower limits, the adiponitrile process stream may comprise greater than 0 wt. %, low-boiling components, e.g., greater than 1 wt %, greater than 2 wt %, greater than 3 wt %, greater than 5 wt. %, greater than 10 wt. %, greater than 12 wt. %, or greater than 15 wt. %.

The adiponitrile process stream also comprises high-boiling components. Generally, the high-boiling components are impurities having relatively high boiling points. For example, each of the high-boiling components may have a boiling point of greater than 395° C., e.g., greater than 400° C., greater than 405° C., greater than 408° C., greater than 410° C., or greater than 415° C. Examples of high-boiling components that may be present in the crude adiponitrile stream include isomeric tricyanohexane, tri(2-cyanoethyl)amine, and combinations thereof.

In one embodiment, the adiponitrile process stream comprises high-boiling components in an amount ranging from 0 wt. % to 50 wt. %, e.g., from 0 wt. % to 40 wt. %, from 0 wt. % to 35 wt. %, from 0 wt. % to 25 wt. %, from 0 wt. % to 20 wt. %, from 0.5 wt. % to 50 wt. %, from 0.5 wt. % to 40 wt. %, from 0.5 wt. % to 35 wt. %, from 0.5 wt. % to 25 wt. %, from 0.5 wt. % to 20 wt. %, from 1 wt. % to 50 wt. %, from 1 wt. % to 40 wt. %, from 1 wt. % to 35 wt. %, from 1 wt. % to 25 wt. %, from 1 wt. % to 20 wt. %, from 2 wt. % to 50 wt. %, from 2 wt. % to 40 wt. %, from 2 wt. % to 35 wt. %, from 2 wt. % to 25 wt. %, from 2 wt. % to 20 wt. %, from 3 wt. % to 50 wt. %, from 3 wt. % to 40 wt. %, from 3 wt. % to 35 wt. %, from 3 wt. % to 25 wt. %, from 3 wt. % to 20 wt. %, from 5 wt % to 15 wt %, from 5 wt. % to 50 wt. %, from 5 wt. % to 40 wt. %, from 5 wt. % to 35 wt. %, from 5 wt. % to 25 wt. %, or from 5 wt. % to 20 wt. %. In terms of upper limits, the adiponitrile process stream may comprise less than 50 wt. % high-boiling components, e.g., less than 40 wt. %, less than 35 wt. %, less than 30 wt. %, less than 25 wt. % or less than 20 wt. %. In terms of lower limits, the adiponitrile process stream may comprise greater than 0 wt. %, e.g., greater than 0.5 wt. %, greater than 1 wt. %, greater than 2 wt. %, greater than 3 wt. %, or greater than 5 wt. %.

In some embodiments, the adiponitrile process stream may also comprise solid impurities. These impurities may include various organic impurities that are solid under the temperature and pressure conditions. For example, the solid impurities may include solid cyanocarbon compounds. In one embodiment, the adiponitrile process stream comprises solid impurities in an amount ranging from 0 wt. % to 25 wt. %, e.g., from 0 wt. % to 20 wt. %, from 0 wt. % to 15 wt. %, or from 0 wt. % to 10 wt. %. In terms of upper limits, the adiponitrile process stream may comprise less than 25 wt. %, e.g., less than 20 wt. %, less than 15 wt. %, or less than 10 wt. %.

In some embodiments, the adiponitrile process stream comprises nitriles (generally, e.g., high boiling point and/or low boiling point nitriles). In one embodiment, the adiponitrile process stream comprises nitriles in an amount ranging from 0 wt. % to 90 wt. %, based on the total weight of the feed stream, e.g., from 0 wt. %, to 89 wt. %, from 0 wt. % to 88 wt. %, from 0 wt. % to 85 wt. %, from 0 wt. % to 84 wt. %, from 10 wt. % to 90 wt. %, from 10 wt. %, to 89 wt. %, from 10 wt. % to 88 wt. %, from 10 wt. % to 85 wt. %, from 10 wt. % to 84 wt. %, from 20 wt. % to 90 wt. %, from 20 wt. %, to 89 wt. %, from 20 wt. % to 88 wt. %, from 20 wt. % to 85 wt. %, from 20 wt. % to 84 wt. %, from 30 wt. % to 90 wt. %, from 30 wt. %, to 89 wt. %, from 30 wt. % to 88 wt. %, from 30 wt. % to 85 wt. %, from 30 wt. % to 84 wt. %, from 40 wt. % to 90 wt. %, from 40 wt. %, to 89 wt. %, from 40 wt. % to 88 wt. %, from 40 wt. % to 85 wt. %, from 40 wt. % to 84 wt. %, from 50 wt. % to 90 wt. %, from 50 wt. %, to 89 wt. %, from 50 wt. % to 88 wt. %, from 50 wt. % to 85 wt. %, or from 50 wt. % to 84 wt. %. In terms of upper limits, the adiponitrile process stream may comprise less than 90 wt. % nitriles, e.g., 89 wt. %., less than 88 wt. %, less than 85 wt. %, or less than 84 wt. %, In terms of lower limits, the adiponitrile process stream may comprise greater than 0 wt. % nitriles, e.g., greater than 10 wt. %, greater than 20 wt. %, greater than 30 wt. %, greater than 40 wt. %, or greater than 50.

### Flashing and Adiponitrile Process Stream

As noted above, the adiponitrile process stream is separated in a flashing step to form the first intermediate adiponitrile stream (an overhead stream) comprising adiponitrile and low-boiling components (lights) and (optionally lower amounts of) high-boiling components (heavies) and a heavies stream comprising high-boiling components and solid impurities. The flashing step, in some cases, removes a significant portion (if not all) of the heavies and/or the solid impurities present in the adiponitrile process stream. The inventors have found that removal of the heavies prior to further processing beneficially reduces the decomposition of the high-boiling components and thereby improves the efficiency of the total purification process. Without this initial removal of heavies, additional non-TCH components are formed, which must then be separated, creating additional operations and uncertainties. Furthermore, the inventors have also found that early removal of the heavies and the solid impurities reduces fouling of columns, which improves downstream efficiency and eliminates or reduces the need for subsequent separation operations. The residence time of the feed stream in the flashing may be a short residence time as discussed herein.

In some embodiments, the separating step includes separation in a flasher, e.g., a flash evaporator. In these embodiments, the adiponitrile process stream is evaporated and separated into an overhead stream e.g., the first intermediate adiponitrile stream, and the (first) bottoms stream. Various flashers are known to those of ordinary skill in the art, and any suitable flasher may be employed as long as the separation described herein is achieved. In some embodiments, the separation in the flasher may be caused by reducing the pressure, e.g., an adiabatic flash, without heating the feed stream. In other embodiments, the separation in the flasher may be caused by raising the temperature of the feed stream without changing the pressure. In still other embodiments, the separation in the flasher may be caused by reducing the pressure while heating the feed stream. In some embodiments, the first separating step is achieved via a wiped film evaporator (WFE).

In some embodiments, the flashing step includes separating the adiponitrile process stream in a flash evaporator at reduced pressure, e.g., under a vacuum. In some embodiments, the pressure in the flash evaporator is reduced to less than 25 torr, e.g., less than 20 torr, less than 10 torr, less than 5 torr, or less than 1 torr. In some embodiments, the flash vessel of the flashing step is kept at a constant temperature. In some embodiments, the temperature of the flash vessel may be from 175° C. to 235° C., e.g., from 180° C. to 230° C., from 185° C. to 225° C., or from 190° C. to 220° C. The first bottoms stream comprises high-boiling components (heavies). Examples of heavies that may be present in the first bottoms stream include isomeric tricyanohexane, tri(2-cyanoethyl)amine, and combinations thereof. In one embodiment, the separation step occurs in a flasher, and the first bottoms stream comprises isomeric tricyanohexane and tri(2-cyanoethyl)amine. The first bottoms stream also may comprise solid impurities. In one embodiment, the flashing step removes all (substantially all) of the solid impurities from the adiponitrile process stream. Said another way, in this embodiment, the flash overhead stream comprises effectively 0 wt. % solid impurities. In other embodiments, the flashing step may remove less than 100% of the solid impurities, e.g., less than 99.9%, less than 99%, or less than 98%.

The first intermediate adiponitrile stream may comprise less than 90 wt % adiponitrile, e.g., less than 75 wt %, less than 50 wt %, less than 40 wt %, less than 35 wt %, less than 30 wt %, less than wt 20%, less than 18 wt %, less than 15 wt %, less than 12 wt %, less than 10 wt %, less than 5 wt %, less than 4 wt %, less than 3 wt %, or less than 2 wt %. In terms of ranges, the first intermediate adiponitrile stream may comprise from 0.1 wt % to 90 wt % adiponitrile, e.g., from 0.1 wt % to 75 wt %, from 0.1 wt % to 40 wt %, from 0.1 wt % to 10 wt %, from 0.1 wt % to 5 wt %, from 0.5 wt % to 5 wt %, from 0.5 wt % to 3 wt %, from 0.5 wt % to 30 wt %, from 1 wt % to 20 wt %, from 2 wt % to 20 wt %, from 5 wt % to 18 wt %, or from 5 wt % to 15 wt %. In terms of lower limits, the first intermediate adiponitrile stream may comprise greater than 0.1 wt % adiponitrile, e.g., greater than 0.3 wt %, greater than 0.5 wt %, greater than 0.7 wt %, greater than 1.0 wt %, greater than 1.5 wt %, greater than 2 wt %, or greater than 5 wt %.

In some embodiments, the first intermediate adiponitrile stream comprises less than 99 wt. % TCH, e.g., less than 97 wt %, less than 90 wt %, less than 80 wt %, less than 70 wt %, less than 50 wt. %, less than 35 wt. %, less than 25 wt. %, less than 20 wt. %, less than 18 wt. %, less than 15 wt. %, less than 12 wt. %, less than 10 wt. %, or less than 5 wt. %. In terms of ranges, the first intermediate adiponitrile stream may comprise from 0.1 wt % to 99 wt % TCH, e.g., from 50 wt % to 99 wt %, from 75 wt % to 98 wt %, from 85 wt % 98 wt %, from 90 wt % to 97 wt %, from 0.1 wt. % to 25 wt. %, from 0.5 wt. % to 23 wt. %, from 0.5 wt. % to 20 wt. %, from 1 wt. % to 15 wt. %, from 1.5 wt. % to 12 wt. %, or from 2 wt. % to 11 wt. %. In terms of lower limits, the first intermediate adiponitrile stream may comprise greater than 0.1 wt. % TCH, e.g., greater than 0.3 wt. %, greater than 0.5 wt. %, greater than 0.7 wt. %, greater than 1.0 wt. %, greater than 1.5 wt. %, greater than 2 wt. %, greater than 5 wt. %, greater than 25 wt %, greater than 50 wt %, greater than 75 wt %, greater than 85 wt %, greater than 85 wt %, or greater than 90 wt %.

In one embodiment, the first intermediate adiponitrile stream comprises lights in an amount ranging from 0 wt. % to 70 wt. %, e.g., from 0.1 wt % to 30 wt %, from 0.1 wt % to 50 wt %, from 0 wt. % to 25 wt. %, from 0 wt. %, to 20 wt. %, from 0 wt. % to 15 wt. %, from 0 wt. % to 10 wt. %, from 1 wt. % to 30 wt. %, from 1 wt. % to 25 wt. %, from 1 wt. %, to 20 wt. %, from 1 wt. % to 15 wt. %, from 1 wt. % to 10 wt. %, from 2 wt. % to 30 wt. %, from 2 wt. % to 25 wt. %, from 2 wt. %, to 20 wt. %, from 2 wt. % to 15 wt. %, from 2 wt. % to 10 wt. %, from 3 wt. % to 30 wt. %, from 3 wt. % to 25 wt. %, from 3 wt. %, to 20 wt. %, from 0.1 wt. %, to 10 wt. %, from 0.1 wt. %, to 5 wt. %, from 0.3 wt. %, to 3 wt. %, from 0.5 wt. %, to 2 wt. %, from 1 wt. %, to 3 wt. %, from 3 wt. % to 15 wt. %, from 3 wt. % to 10 wt. %, from 4 wt. % to 30 wt. %, from 4 wt. % to 25 wt. %, from 4 wt. %, to 20 wt. %, from 4 wt. % to 15 wt. %, from 4 wt. % to 10 wt. %, from 5 wt. % to 30 wt. %, from 5 wt. % to 25 wt. %, from 5 wt. %, to 20 wt. %, from 5 wt. % to 15 wt. %, or from 5 wt. % to 10 wt. %. In terms of upper limits, the first intermediate adiponitrile stream may comprise less than 70 wt. % lights, e.g., less than 50 wt %, less than 30 wt %, less than 25 wt. %, less than 20 wt. %, less than 15 wt. %, less than 10 wt. %, less than 5 wt %, less than 3 wt %, or less than 2 wt %. In terms of lower limits, the first intermediate adiponitrile stream may comprise greater than 0 wt. % lights, e.g., greater than 0.1 wt %, greater than 0.3 wt %, greater than 0.5 wt %, greater than 1 wt. %, greater than 2 wt. %, greater than 3 wt. %, greater than 4 wt. %, or greater than 5 wt. %.

In one embodiment, the first intermediate adiponitrile stream comprises heavies in an amount ranging from 0 wt. % to 20 wt. %, e.g., from 0 wt. % to 15 wt. %, from 0 wt. % to 10 wt. %, from 0 wt. % to 8 wt. %, from 0 wt. % to 5 wt. %, from 0.5 wt. % to 20 wt. %, from 0.5 wt. % to 15 wt. %, from 0.5 wt. % to 10 wt. %, from 0.5 wt. % to 8 wt. %, from 0.5 wt. % to 5 wt. %, from 1 wt. % to 20 wt. %, from 1 wt. % to 15 wt. %, from 1 wt. % to 10 wt. %, from 1 wt. % to 8 wt. %, from 1 wt. % to 5 wt. %, from 1.5 wt. % to 20 wt. %, from 1.5 wt. % to 15 wt. %, from 1.5 wt. % to 10 wt. %, from 1.5 wt. % to 8 wt. %, from 1.5 wt. % to 5 wt. %, from 2 wt. % to 20 wt. %, from 2 wt. % to 15 wt. %, from 2 wt. % to 10 wt. %, from 2 wt. % to 8 wt. %, from 2 wt. % to 5 wt. %, from 2.5 wt. % to 20 wt. %, from 2.5 wt. % to 15 wt. %, from 2.5 wt. % to 10 wt. %, from 2.5 wt. % to 8 wt. %, or from 2.5 wt. % to 5 wt. %. In terms of upper limits, the first intermediate adiponitrile stream may comprise less than 20 wt. % heavies, e.g., less than 15 wt. %, less than 10 wt. %, less than 8 wt. %, or less than 5 wt. %. In terms of lower limits, the first intermediate adiponitrile stream may comprise greater than 0 wt. % heavies, e.g., greater than 0.5 wt. %, greater than 1 wt. %, greater than 1.5 wt. %, greater than 2 wt. %, or greater than 2.5 wt. %.

In some cases, the flashing step removes a significant portion of the heavies from the first intermediate adiponitrile stream. Said another, the adiponitrile process stream comprises low amounts, if any, of the heavies initially present in the feed stream. In some embodiments, the first intermediate adiponitrile stream comprises less than 70% of the heavies present in the feed stream, e.g., less than 65%, less than 60%, less than 55%, or less than 50%.

### Separation and First TCH Stream

In some embodiments, the (first) intermediate adiponitrile stream may be separated in a separating step to form the (second) intermediate adiponitrile stream comprising adiponitrile and lights (low-boiling components), optionally a first TCH stream, and a heavies stream comprising heavies (high-boiling components). In some cases, the separating step may simply separate the adiponitrile process stream, optionally in one or more (distillation) columns, to form the intermediate adiponitrile stream. The separating step, in some cases, removes a significant portion (if not all) of the low-boiling components and high-boiling components present in the intermediate adiponitrile process stream. In some cases, the separating step comprises one or more columns, e.g., two columns. In some embodiments, the separating step comprise two columns and the first distillation column forms a lights stream as an overhead stream (second intermediate adiponitrile stream) and a second bottoms stream. The second bottoms stream is then separated in a second distillation column to form the heavies stream as a third bottoms stream and the TCH stream as a third overhead stream.

The various separating steps discussed herein may include separation of the (first) intermediate adiponitrile stream in one or more distillation columns and/or in one or more flash evaporators. The structure of the one or more distillation columns may vary widely. Various distillation columns are known to those of ordinary skill in the art, and any suitable column may be employed in the second separation step as long as the separation described herein is achieved. For example, the distillation column may comprise any suitable separation device or combination of separation devices. For example, the distillation column may comprise a column, e.g., a standard distillation column, a packed column, an extractive distillation column and/or an azeotropic distillation column. Similarly, as noted above, various flashers are known to those of ordinary skill in the art, and any suitable flasher may be employed in the second separation step as long as the separation described herein is achieved. For example, the flasher may comprise an adiabatic flash evaporator, a heated flash evaporator, or a wipe film evaporator, or combinations thereof.

Embodiments of the separating step may include any combination of one or more distillation columns and/or one or more flashers, as long as the aforementioned streams are formed.

In one embodiment, for example, the separating step comprises separating the (first) intermediate adiponitrile stream in two consecutive distillation columns. In this embodiment, the first overhead lights stream is separated in a first distillation column. A second overhead lights stream is collected from the overhead (e.g., the column top and/or a relatively high side draw) of the first distillation column, and a second bottom (intermediate) heavies stream is collected from the bottom (e.g., the column bottom and/or a relatively low side draw) of the first distillation column. At least a portion of the second bottom (intermediate) heavies stream is then separated in a second distillation column. A third bottom heavies stream is collected from the bottom (e.g., the column bottom and/or a relatively low side draw) of the second distillation column. The TCH stream is collected from the overhead (e.g., column top and/or a relatively high side draw) of the second distillation column, e.g., as a third overhead lights stream.

In another embodiment, the separating step comprises separating the (first) intermediate adiponitrile stream in a distillation column and an evaporator (e.g., flasher, WFE, or falling film evaporator). In this embodiment, the first distillation column is separated in a first distillation column. A second overhead lights stream is collected from the overhead (e.g., the column top and/or a relatively high side draw) of the first distillation column, a second bottom heavies stream is collected from the bottom (e.g., the column bottom and/or a relatively low side draw) of the first distillation column, and a side draw is collected is a side cut of the first distillation column. At least a portion of the side draw is then separated draw in an evaporator. A third overhead lights stream is collected from the top of the evaporator, and the TCH stream is collected from the bottom of the evaporator, e.g., as a third bottom heavies stream.

In another embodiment, the separating step comprises separating the (first) intermediate adiponitrile stream in a three distillation columns. In this embodiment, the first overhead lights stream is separated in a first distillation column. A second overhead lights stream is collected from the overhead (e.g., the column top and/or a relatively high side draw) of the first distillation column, and a second bottom heavies stream is collected from the bottom (e.g., the column bottom and/or a relatively low side draw) of the first distillation column. At least a portion of the second bottom heavies stream is then separated in a second distillation column. A third overhead lights stream is collected from the overhead (e.g., the column top and/or a relatively high side draw) of the second distillation column, and third bottom heavies stream is collected from the bottom (e.g., the column bottom and/or a relatively low side draw) of the second distillation column. At least a portion of the third overhead lights stream is then separated in a third distillation column. A fourth bottom heavies stream is collected from the bottom (e.g., the column bottom and/or a relatively low side draw) of the third distillation column, and the TCH stream is collected from the top (e.g., the column top and/or a relatively high side draw) of the third distillation column, e.g., as a fourth overhead lights stream.

In another embodiment, the separating step comprises separating the (first) intermediate adiponitrile stream in a two distillation columns and an evaporator (e.g., flasher, WFE, or falling film evaporator). In this embodiment, the first overhead lights stream is separated in a first distillation column. A second overhead lights stream is collected from the overhead (e.g., the column top and/or a relatively high side draw) of the first distillation column, and a second bottom heavies stream is collected from the bottom (e.g., the column bottom and/or a relatively low side draw) of the first distillation column. At least a portion of the second bottom heavies stream is then separated in a second distillation column. A third overhead lights stream is collected from the overhead (e.g., the column top and/or a relatively high side draw) of the second distillation column, and third bottom heavies stream is collected from the bottom (e.g., the column bottom and/or a relatively low side draw) of the second distillation column. At least a portion of the third overhead lights stream is then separated in an evaporator. A fourth overhead lights stream is collected from the top of the evaporator, and the TCH stream is collected from the bottom of the evaporator, e.g., as a fourth bottom heavies stream.

### Adiponitrile Stream

In some embodiments, the (second) intermediate adiponitrile stream may comprise greater than 1 wt % adiponitrile, e.g., greater than 5 wt %, greater than 6 wt %, greater than 10 wt %, greater than 20 wt %, greater than 25 wt %, greater than 30 wt %, greater than 35 wt %, or greater than 50 wt %. In terms of ranges, the intermediate adiponitrile stream may comprise from 1 wt % to 95 wt % adiponitrile, from 5 wt % to 95 wt %, from 7 wt % to 75 wt %, from 5 wt % to 35 wt %, from 6 wt % to 30 wt %, from 25 wt % to 75 wt %, from 30 wt % to 70 wt %, or from 40 wt % to 60 wt %. In terms of lower limits, the intermediate adiponitrile stream comprises less than 95 wt % TCH, e.g., less than wt 90%, less than 85 wt %, less than 80 wt %, less than 75 wt %, less than 65 wt %, less than 60 wt %, or less than 30 wt %.

In some embodiments, the (second) intermediate adiponitrile stream may comprise greater than 1 wt % TCH, e.g., greater than 5 wt %, greater than 10 wt %, greater than 20 wt %, greater than 25 wt %, greater than 30 wt %, greater than 35 wt %, greater than 50 wt %, greater than 60 wt %, or greater than 70 wt %. In terms of ranges, the intermediate adiponitrile stream may comprise from 1 wt % to 95 wt % TCH, from 5 wt % to 95 wt %, from 20 wt % to 95 wt %, from 30 wt % to 95 wt %, from 45 wt % to 80 wt %, from 50 wt % to 95 wt %, from 60 wt % to 90 wt %, from 70 wt % to 90 wt %, from 25 wt % to 75 wt %, from 30 wt % to 70 wt %, or from 40 wt % to 60 wt %. In terms of lower limits, the intermediate adiponitrile stream comprises less than 95 wt % TCH, e.g., less than wt 90%, less than 85 wt %, less than 80 wt %, less than 75 wt %, less than 65 wt %, or less than 60 wt %.

The (second) intermediate adiponitrile stream may comprise less than 70 wt % lights, e.g., less than 50 wt %, less than 35 wt %, less than 25 wt %, less than 20 wt %, less than 15 wt %, less than 12 wt %, or less than 10 wt %. In terms of ranges, the intermediate adiponitrile stream may comprise from 0.1 wt % to 70 wt % lights, e.g., from 0.1 wt % to 50 wt %, from 0.1 wt % to 25 wt %, from 0.5 wt % to 25 wt %, from 10 wt % to 25 wt %, from 1 wt % to 20 wt %, from 2 wt % to 18 wt %, from 2 wt % to 15 wt %, or from 2 wt % to 10 wt %. In terms of lower limits, the intermediate adiponitrile stream may comprise greater than 0.1 wt % lights, e.g., greater than 0.3 wt %, greater than 0.5 wt %, greater than 0.7 wt %, greater than 1.0 wt %, greater than 1.5 wt %, greater than 2 wt %, or greater than 5 wt %. As noted above, in some cases, the term “lights” refers to components that have lower boiling points, e.g., lower boiling points than adiponitrile or lower boiling points than TCH.

The (second) intermediate adiponitrile stream comprises high-boiling components (heavies). In one embodiment, the (second) intermediate adiponitrile stream comprises high-boiling components in an amount ranging from 0.1 wt % to 50 wt %, e.g., from 0.1 wt. % to 20 wt. %, from 0.1 wt. % to 10 wt. %, from 0.5 wt. % to 10 wt. %, from 0.5 wt. % to 5 wt. %, from 1 wt. % to 3 wt. %, from 5 wt. % to 50 wt. %, e.g., from 5 wt. % to 45 wt. %, from 5 wt. % to 40 wt. %, from 5 wt. % to 35 wt. %, from 5 wt. % to 30 wt. %, from 8 wt. % to 50 wt. %, from 8 wt. % to 45 wt. %, from 8 wt. % to 40 wt. %, from 8 wt. % to 35 wt. %, from 8 wt. % to 30 wt. %, from 10 wt. % to 50 wt. %, from 10 wt. % to 45 wt. %, from 10 wt. % to 40 wt. %, from 10 wt. % to 35 wt. %, from 10 wt. % to 30 wt. %, from 12 wt. % to 50 wt. %, from 12 wt. % to 45 wt. %, from 12 wt. % to 40 wt. %, from 12 wt. % to 35 wt. %, from 12 wt. % to 30 wt. %, from 15 wt. % to 50 wt. %, from 15 wt. % to 45 wt. %, from 15 wt. % to 40 wt. %, from 15 wt. % to 35 wt. %, or from 15 wt. % to 30 wt. %. In terms of upper limits, the (second) intermediate adiponitrile stream may comprise less than 50 wt. % high-boiling components, e.g., less than 45 wt. %, less than 40 wt. %, less than 35 wt. %, less than 30 wt. %, less than 20 wt. %, less than 10 wt. %, less than 5 wt. %, or less than 3 wt. %. In terms of lower limits, the (second) intermediate adiponitrile stream may comprise greater than 0.1 wt. % high-boiling components, e.g., greater than 0.5 wt %, greater than 1 wt. %, greater than 5 wt. %, greater than 8 wt. %, greater than 10 wt. %, greater than 12 wt. %, or greater than 15 wt. %.

In some cases, the separation of the first intermediate adiponitrile stream may be achieved in a two column system. The first column yields the second intermediate adiponitrile stream and an intermediate bottoms stream, which is fed to the second column. The intermediate bottoms stream may comprise high amounts of TCH and may then be further separated, e.g., in one or more additional columns. For example, the intermediate bottoms stream, in some embodiments, comprises TCH in high amounts ranging from 90 wt. % to 100 wt. %, e.g., from 90 wt. % to 99.9 wt. %, from 90 wt. % to 99 wt. %, from 90 wt. % to 98 wt. %, from 92.5 wt. % to 100 wt. %, from 92.5 wt. % to 99.9 wt. %, from 92.5 wt. % to 99 wt. %, from 92.5 to 98 wt. %, from 95 wt. % to 100 wt. %, from 95 wt. % to 99.9 wt. %, from 95 wt. % to 99 wt. %, from 95 to 98 wt. %, from 97.5 wt. % to 100 wt. %, from 97.5 wt. % to 99.9 wt. %, from 97.5 to 99 wt. %, or from 97.5 to 98 wt. %. In terms of upper limits, the intermediate bottoms stream may comprise less than 100 wt. % TCH, e.g., less than 99.9 wt. % less than 99 wt. %, or less than 98 wt. %. In terms of lower limits, the intermediate bottoms stream may comprise greater than 90 wt. %, e.g., greater than 92.5 wt. %, greater than 95 wt. %, or greater than 97.5 wt. %.

The intermediate bottoms stream may further comprise small amounts of adiponitrile and lights (amounts similar to those discussed herein for the TCH stream). The intermediate bottoms stream may further comprise heavies (amounts similar to those discussed herein for the (second) intermediate adiponitrile stream.

In some case, the intermediate bottoms stream may be further separated, e.g., to yield the bottoms heavies stream and the TCH stream.

### TCH Stream

As a result of the disclosed operation parameters, in some embodiments, the (first) TCH stream may comprise greater than 1 wt % TCH, e.g., greater than 5 wt %, greater than 10 wt %, greater than 20 wt %, greater than 25 wt %, greater than 30 wt %, greater than 35 wt %, greater than 50 wt %, greater than 75 wt %, greater than 85 wt %, greater than 90 wt %, greater than 93%, or greater than 95 wt %. In terms of ranges, the first TCH stream may comprise from 1 wt % to 99.9 wt % TCH, e.g., from 25 wt % to 99.9 wt %, from 50 wt % to 99.9 wt %, from 75 wt % to 99.9 wt %, from 90 wt % to 99.9 wt %, from 85 wt % to 99.5 wt %, from 5 wt % to 99 wt %, from 50 wt % to 99 wt %, from 5 wt % to 95 wt %, from 25 wt % to 90 wt %, from 45 wt % to 90 wt %, or from 50 wt % to 85 wt %. In terms of upper limits, the first TCH stream comprises less than 99.9 wt % TCH, e.g., less than 99 wt %, less than 99.5 wt %, less than 95 wt %, less than wt 90%, less than 85 wt %, less than 80 wt %, less than 75 wt %, or less than 65 wt %.

In some embodiments, the (first) TCH stream comprises TCH in higher amounts ranging from 90 wt. % to 100 wt. %, e.g., from 90 wt. % to 99.9 wt. %, from 90 wt. % to 99 wt. %, from 90 wt. % to 98 wt. %, from 92.5 wt. % to 100 wt. %, from 92.5 wt. % to 99.9 wt. %, from 92.5 wt. % to 99 wt. %, from 92.5 to 98 wt. %, from 95 wt. % to 100 wt. %, from 95 wt. % to 99.9 wt. %, from 95 wt. % to 99 wt. %, from 95 to 98 wt. %, from 97.5 wt. % to 100 wt. %, from 97.5 wt. % to 99.9 wt. %, from 97.5 to 99 wt. %, or from 97.5 to 98 wt. %. In terms of upper limits, the TCH stream may comprise less than 100 wt. % TCH, e.g., less than 99.9 wt. % less than 99 wt. %, or less than 98 wt. %. In terms of lower limits, the TCH stream may comprise greater than 90 wt. %, e.g., greater than 92.5 wt. %, greater than 95 wt. %, or greater than 97.5 wt. %. Conventional processes have been unable to achieve such high TCH purity levels.

In one embodiment, the TCH stream comprises impurities, e.g., heavies and/or lights, in an amount ranging from 0 wt. % to 10 wt. %, e.g., from 0 wt. % to 7.5 wt. %, from 0 wt. % to 5 wt. %, from 0 wt. % to 2.5 wt. %, from 0.1 wt. % to 10 wt. %, from 0.1 wt. % to 7.5 wt. %, from 0.1 wt. % to 5 wt. %, from 0.1 wt. % to 2.5 wt. %, 0.1 wt. % to 1.5 wt. %, 0.2 wt. % to 1.2 wt. %, 0.3 wt. % to 1.5 wt. %, 0.5 wt. % to 1.0 wt. %, from 1 wt. % to 10 wt. %, from 1 wt. % to 7.5 wt. %, from 1 wt. % to 5 wt. %, from 1 wt. % to 2.5 wt. %, from 2 wt. % to 10 wt. %, from 2 wt. % to 7.5 wt. %, from 2 wt. % to 5 wt. %, or from 2 wt. % to 2.5 wt. %. In terms of upper limits, the TCH stream may comprise less than 10 wt. % impurities, e.g., less than 7.5 wt. %, less than 5 wt. %, less than 2.5 wt. %, less than 1.5 wt. %, less than 1.2 wt. %, or less than 1.0 wt. %. In terms of lower limits, the TCH stream may comprise greater than 0 wt. % impurities, e.g., greater than 0.1 wt. %, greater than 1 wt. %, or greater than 2 wt. %. The TCH stream may comprise amines and/or nitriles in these amounts. In some cases, the use of lower pressures in the separation surprisingly provides for improved separation of components having boiling points close to that of TCH, e.g., CVA. These ranges and limits apply to heavies and lights individually or combined.

The (first) TCH stream may comprise less than 25 wt. % adiponitrile, e.g., less than 23 wt. %, less than 20 wt. %, less than 18 wt. %, less than 15 wt. %, less than 12 wt. %, less than 10 wt. %, less than 8 wt. %, less than 5 wt. %, less than 3 wt. %, less than 1 wt. %, less than 0.05 wt. %, or less than 0.03 wt. %. In terms of ranges, the (first) TCH stream may comprise from 0.001 wt. % to 25 wt. % adiponitrile, e.g., from 0.05 wt. % to 5 wt. %, from 0.1 wt. % to 25 wt. %, from 0.5 wt. % to 22 wt. %, from 1 wt. % to 20 wt. %, from 2 wt. % to 20 wt. %, or from 5 wt. % to 18 wt. %. In terms of lower limits, the (first) TCH stream may comprise greater than 0.001 wt. % adiponitrile, e.g., greater than 0.01 wt %, greater than 0.01 wt. %, greater than 0.5 wt. %, greater than 1.0 wt. %, greater than 2.0 wt. %, greater than 5.0 wt. %, greater than 10 wt. %, or greater than 15 wt. %.

In one embodiment, the TCH stream comprises from 0 wt. % to 0.05 wt. % adiponitrile, from 0 wt. % to 0.1 wt. % di(2-cyanoethyl) amine, from 0 wt. % to 0.05 wt. % cyanovaleramide, and from 0 wt. % to 0.05 wt. % tri(2-cyanoethyl) amine.

### Heavies Stream

As a result of the disclosed operation parameters, in some embodiments, the heavies stream, which may, in some cases be a bottoms stream from a second column of a two column system, may comprise high amounts of TCH as well as heavies. In some cases, the heavies stream may comprise TCH in amounts ranging from 90 wt. % to 100 wt. %, e.g., from 90 wt. % to 99.9 wt. %, from 90 wt. % to 99 wt. %, from 90 wt. % to 98 wt. %, from 92.5 wt. % to 100 wt. %, from 92.5 wt. % to 99.9 wt. %, from 92.5 wt. % to 99 wt. %, from 92.5 to 98 wt. %, from 95 wt. % to 100 wt. %, from 95 wt. % to 99.9 wt. %, from 95 wt. % to 99 wt. %, from 95 to 98 wt. %, from 97.5 wt. % to 100 wt. %, from 97.5 wt. % to 99.9 wt. %, from 97.5 to 99 wt. %, or from 97.5 to 98 wt. %. In terms of upper limits, the heavies stream may comprise less than 100 wt. % TCH, e.g., less than 99.9 wt. % less than 99 wt. %, or less than 98 wt. %. In terms of lower limits, the heavies stream may comprise greater than 90 wt. %, e.g., greater than 92.5 wt. %, greater than 95 wt. %, or greater than 97.5 wt. %.

In some embodiments, the heavies stream may comprise low amounts of lights and/or adiponitrile. For example, the heavies stream may comprise lights and/or adiponitrile in amounts similar to those discussed above with respect to the intermediate bottoms stream or the TCH stream. The heavies stream may further comprise heavies. The heavies stream may further comprise heavies in amounts similar to those discussed herein for the (second) intermediate adiponitrile stream.

### Purification

In some cases, the intermediate adiponitrile stream is purified, optionally via one or more distillation columns, to form a purified adiponitrile stream comprising at greater than 50 wt % adiponitrile. In some cases, the intermediate adiponitrile stream may be purified using existing purification equipment outside of the process, e.g., in a separation train for a different process.

In some embodiments, the purified adiponitrile stream comprises greater than 10 wt % adiponitrile, e.g., greater than 25 wt %, greater than 50 wt %, greater than 75 wt %, greater than 90 wt %, greater than 92 wt %, greater than 95 wt %, or greater than 97 wt %. In terms of ranges, the purified adiponitrile stream may comprise from 50 wt % to 100 wt % adiponitrile, e.g., from 50 wt % to 99.5 wt %, from 65 wt % to 99 wt %, from 75 wt % to 99 wt %, from 90 wt % to 97 wt %, or from 90 wt % to 95 wt %.

In some cases, both the purified adiponitrile stream and the TCH stream exist (as described herein). In some embodiments, the purified adiponitrile stream comprises greater than 95 wt % adiponitrile and the TCH stream comprises greater than 95 wt % TCH.

In some cases, the purification of the intermediate adiponitrile stream may be conducted in an outside system, e.g., a refinement process, for example in an adiponitrile production process.

### Decomposition

As noted above, the inventors now have found that, in conventional adiponitrile purification processes, certain high-boiling components are prone to decomposition into impurities having both higher boiling points and/or lower boiling points. The inventors have also found that even TCH can decompose at high pressures and/or temperatures in conventional processes. In particular, the inventors have now found that prolonged exposure to high pressures and/or temperatures, such as in columns, contributes to the decomposition of high-boiling components. By utilizing the specific process parameters disclosed herein, this decomposition can be effectively mitigated. In particular, the use of lower pressures provides for reduction or elimination of decomposition products.

Conventional processes typically require the exposing process streams to high temperatures due to the presence of high-boiling components. TCH, for example, of about 407° C. at atmospheric pressure. As can be appreciated by those skilled in the art, purification of TCH therefore conventionally requires exposing process streams to high temperatures, e.g., at least 350° C., at least 375° C., at least 400° C., or at least 410° C. At these high temperatures, however, the present inventors have found that high-boiling components, such as TCH and adiponitrile, rapidly decompose. As a result, conventional processes experience high inefficiencies. By utilizing the specific process parameters disclosed herein, however, this decomposition can be effectively mitigated or eliminated.

In one aspect, the purification process may inhibit decomposition by reducing the residence time during which process streams are exposed to high temperatures, e.g., in a separation operation. Generally, process streams may be exposed to high temperatures and/or pressures in a column. In order to reduce prolonged exposure, the process may reduce the residence time of a stream in a given column (or flasher). For example, the process may control the residence time of the (first or second) intermediate adiponitrile stream or the TCH stream (or another purification stream) in a column. In one embodiment, the process limits the residence time of the (first or second) intermediate adiponitrile stream or the TCH stream (or another purification stream) in a column to less than 8 hours, e.g., less than 7 hours, less than 6 hours, less than 5 hours, or less than 4 hours.

In some aspects, the purification processes may inhibit decomposition by reducing the exposure of process streams to high pressures and/or pressure drops. For example, the process may control the pressure to which the adiponitrile process stream (or another purification stream) is exposed, e.g., in the separation step. In one embodiment, the purification process limits the pressure at which separation step(s) are conducted. For example, operation pressure may be limited to less than 50 torr, e.g., less than 45 torr, less than 40 torr, less than 35 torr, less than 30 torr, or less than 25 torr. In order to reduce prolonged exposure to high pressures, the process may reduce the residence time of a stream in a given column (or flasher). For example, the process may control the residence time of the (first or second) intermediate adiponitrile stream or the TCH stream in a high-pressure column (e.g., a column with a pressure greater than 50 torr).

In one aspect, the separation and/or purification steps may inhibit decomposition by reducing the exposure of process streams to high temperatures. For example, the process may control the temperature to which the (first or second) intermediate adiponitrile stream of the TCH stream (or another purification stream) is exposed, e.g., in a separation step. In one embodiment, the purification process limits the temperature at which separation step(s) are conducted. For example, operation temperature may be limited to less than 350° C., e.g., less than 325° C., less than 300° C., less than 275° C., or less than 250° C., In terms of ranges operation temperature may range from 225° C. to 350° C., e.g., from 250° C. to 325° C. or from 275° C. to 300° C., or from 250° C. to 275° C.

In some aspects, the process may control both the temperature to which a stream is exposed and the time for which it is exposed to that temperature. For example, the process may control the residence time of the (first or second) intermediate adiponitrile stream or the TCH stream (or another purification stream) in a column as well as the temperature of that distillation column. In one embodiment, the residence time of a stream in temperatures above 230° C. is less than 8 hours. The aforementioned ranges and limits for temperature and residence time may be combined with one another.

In some aspects, the process may control both the temperature to which a stream is exposed and the pressure to which it is exposed. In one embodiment, the process may be controlled such that the stream is not exposed to temperatures above 300° C. or pressures above 35 torr.

In other aspects, the process may inhibit decomposition by utilizing columns with certain physical features. In particular, the distillation columns employed in the purification process may have certain shapes. In some embodiments, the distillation columns have relatively small sumps to minimize exposure to high temperatures. In these embodiments, the sumps of each column may taper to a smaller diameter, which allows or reduced exposure to higher temperatures.

These modifications to conventional purification processes reduce the decomposition of high-boiling components. In some embodiments, these modifications reduce the amount high-boiling components in the first overhead stream that decompose during the second separating step. In one embodiment, the amount of high-boiling components in the (first or second) intermediate adiponitrile stream or the TCH stream (or another purification stream) that decompose is less than 50 wt. % of the high-boiling components in the stream, e.g., less than 45 wt. %, less than 40 wt. %, or less than 30 wt. %. In terms of lower limits, the amount of high-boiling components that decompose may be greater than 0 wt. % of the high-boiling components in the stream, e.g., greater than 5 wt. %, greater than 10 wt. %, or greater than 15 wt. %. In terms of ranges, the amount of high-boiling components that decompose may be from 0 wt. %. to 50 wt. %, e.g., from 0 wt. % to 45 wt. %, from 0 wt. % to 40 wt. %, from 0 wt. % to 30 wt. %, from 5 wt. % to 50 wt. %, from 5 wt. % to 45 wt. %, from 5 wt. % to 40 wt. %, from 5 wt. % to 30 wt. %, from 10 wt. % to 50 wt. %, from 10 wt. % to 45 wt. %, from 10 wt. % to 40 wt. %, from 10 wt. % to 30 wt. %, from 15 wt. % to 50 wt. %, from 15 wt. % to 45 wt. %, from 15 wt. % to 40 wt. %, or from 15 wt. % to 30 wt. %.

In some embodiments, the various process streams individually comprise less than 1 wt % decomposition products of high-boiling components, e.g., less than 0.8 wt %, less than 0.5 wt %, less than 0.3 wt %, less than 0.1 wt %, less than 0.05 wt %, or less than 0.01 wt %.

In some embodiments, the decomposition products will be present in the various bottoms streams, e.g., the bottoms stream of the second distillation column. For examples the bottoms stream(s) may comprise greater than 0.1 wt % decomposition products, e.g., greater than 0.5 wt %, greater than 1.0 wt %, greater than 3.0 wt %, greater than 5.0 wt %, greater than 10.0 wt %, greater than 25.0 wt %, or greater than 50.0 wt %.

As noted above, the high-boiling components may decompose into other high-boiling impurities and/or into low-boiling impurities. In some cases, the high-boiling components may decompose into other high-boiling impurities that were not otherwise present in the system. Said another way, the decomposition may cause the total number of high-boiling impurity compounds in the system to increase. By inhibiting decomposition, as described herein, the increase in the total number of high-boiling impurity compounds present in the system, caused by decomposition, may be reduced.

In some cases, the first column (and/or any of the subsequent purification columns) may operate with a short residence time. The residence time of feed streams in the individual separation and/or purification operations of the process is minimized, e.g., less than 8 hours, e.g., less than 7 hours, less than 6 hours, less than 5 hours, or less than 4 hours. The lower residence times (optionally in combination with the lower pressure drop) unexpectedly contributes to the separation/purification efficiencies.

### Recycle Step

In some embodiments, the process comprises a recycle step of recycling at least a portion of a (bottoms or heavies) stream formed during the separation steps to a point upstream (target). For example, the recycling step may comprise recycling at least a portion of the heavies stream of one of the columns or flashers to a point upstream in the process. In some embodiments, the recycling step comprises recycling at least a portion of the heavies stream of the separation step to the flasher overhead stream of the flashing step. In some embodiments, the recycling step comprises recycling at least a portion of the a bottoms stream of the purification step to the flasher overhead stream of the flashing step and/or the bottoms stream of the separation step.

In one embodiment, the recycled stream comprises heavies, and the concentration of these heavies surprisingly affects the purity of the resultant TCH stream and may help to control the concentration of high-boiling components in the overhead streams to be from 0 wt. % to 10 wt. %. In some cases, the concentration of high-boiling components in the recycle streams leads to lesser amounts of high-boiling components in the various overhead streams, which in turn leads to higher purity of adiponitrile and/or TCH.

In some cases, the recycled stream comprises heavies in an amount ranging from 0 wt. % to 40 wt. %, e.g., from 0 wt. % to 37.5 wt. %, from 0 wt. % to 35 wt. %, from 0 wt. % to 32.5 wt. %, from 0 wt. % to 30 wt. %, from 5 wt. % to 40 wt. %, from 5 wt. % to 37.5 wt. %, from 5 wt. % to 35 wt. %, from 5 wt. % to 32.5 wt. %, from 5 wt. % to 30 wt. %, from 10 wt. % to 40 wt. %, from 10 wt. % to 37.5 wt. %, from 10 wt. % to 35 wt. %, from 10 wt. % to 32.5 wt. %, from 10 wt. % to 30 wt. %, from 15 wt. % to 40 wt. %, from 15 wt. % to 37.5 wt. %, from 15 wt. % to 35 wt. %, from 15 wt. % to 32.5 wt. %, from 15 wt. % to 30 wt. %, from 20 wt. % to 40 wt. %, from 20 wt. % to 37.5 wt. %, from 20 wt. % to 35 wt. %, from 20 wt. % to 32.5 wt. %, or from 20 wt. % to 30 wt. %. In terms of upper limits, the recycled stream may comprise less than 40 wt. % high-boiling components, e.g., less than 37.5 wt. %, less than 35 wt. %, less than 32.5 wt. %, or less than 30 wt. %. In terms of lower limits, the recycled stream may comprise greater than 0 wt. % high-boiling components, e.g., greater than 5 wt. %, greater than 10 wt. %, greater than 15 wt. %, or greater than 20 wt. %.

In some aspects, the recycle step controls the concentration of heavies in the target. For example, the recycle step may control the concentration of the heavies in the flasher overhead stream by recycling a stream containing heavies to the flasher stream.

In one embodiment, due to the recycling, the recycle step controls the concentration of heavies in the target to be from 0 wt. % to 10 wt. %, e.g., from 0 wt. % to 9 wt. %, from 0 wt. % to 8 wt. %, from 0 wt. % to 7 wt. %, from 1 wt. % to 10 wt. %, from 1 wt. % to 9 wt. %, from 1 wt. % to 8 wt. %, from 1 wt. % to 7 wt. %, from 2 wt. % to 10 wt. %, from 2 wt. % to 9 wt. %, from 2 wt. % to 8 wt. %, from 2 wt. % to 7 wt. %, from 3 wt. % to 10 wt. %, from 3 wt. % to 9 wt. %, from 3 wt. % to 8 wt. %, or from 3 wt. % to 7 wt. %. In terms of upper limits, the recycle step may control the concentration of heavies in the target to be less than 10 wt. %, e.g., less than 9 wt. %, less than 8 wt. %, or less than 7 wt. %. In terms of lower limits, the recycle step may control the concentration of heavies in the target to be greater than 0 wt. %, e.g., greater than 1 wt. %, greater than 2 wt. %, or greater than 3 wt. %.

Exemplary separation and/or purification schemes are disclosed in U.S. Provisional Patent No. 62/852,604, filed on May 24, 2019, the contents of which are incorporated by reference herein.

### Configurations

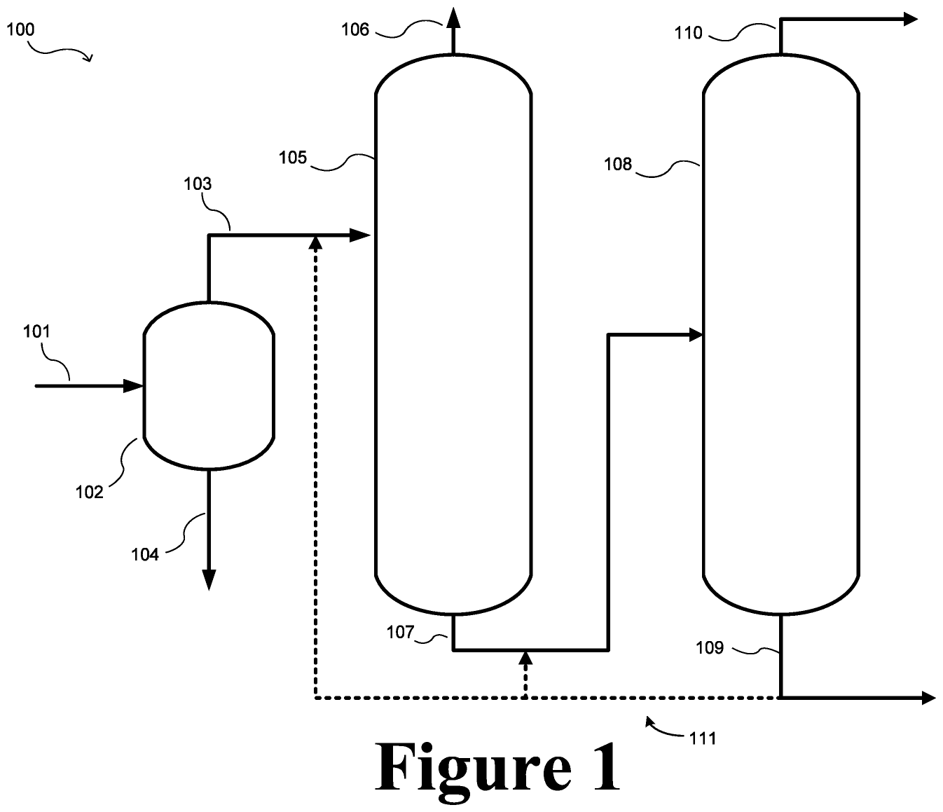

FIGS. 1-5 show schematic overviews of several configurations of the TCH purification processes disclosed herein.

FIG. 1 shows one embodiment of the adiponitrile separation process 100. In this embodiment, an adiponitrile process stream 101 is separated in a flash evaporator 102 to form a first overhead stream 103 and a first bottoms stream 104. The first overhead stream 103 is then separated in a first distillation column 105 to form a lights stream as a second overhead stream 106 and a second bottoms stream 107. The second bottoms stream is then separated in a second distillation column 108 to form a heavies stream as a third bottoms stream 109 and a TCH stream as a third overhead stream 110. This embodiment also features an optional recycle step 111, whereby a portion of the third bottoms stream 109 is recycled to the first overhead stream 103 and/or the second bottoms stream 107.

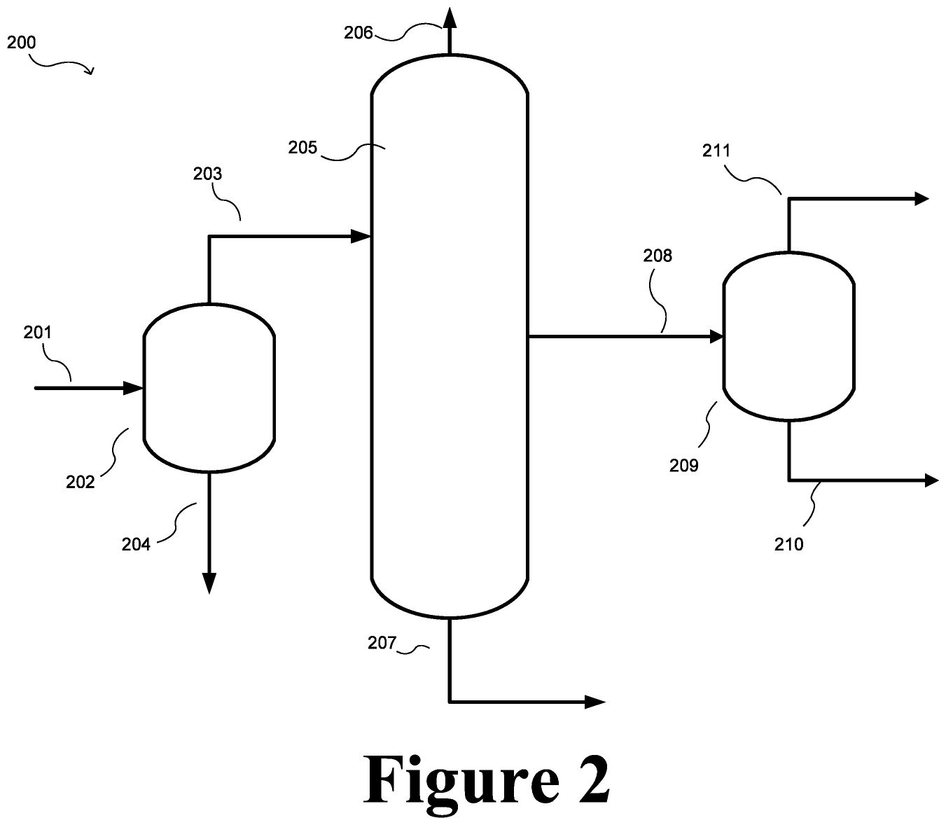

FIG. 2 shows another embodiment of the adiponitrile separation process 200. In this embodiment, an adiponitrile process stream 201 is separated in a flash evaporator 202 to form a first overhead stream 203 and a first bottoms stream 204. The first overhead stream 203 is then separated in a first distillation column 205 to form a lights stream as a second overhead stream 206, a second bottoms stream 207, and a side draw 208. The side draw 208 is then separated in separated in a flasher 209 to form a TCH stream as a third bottoms stream 210 and a third overhead stream 211.

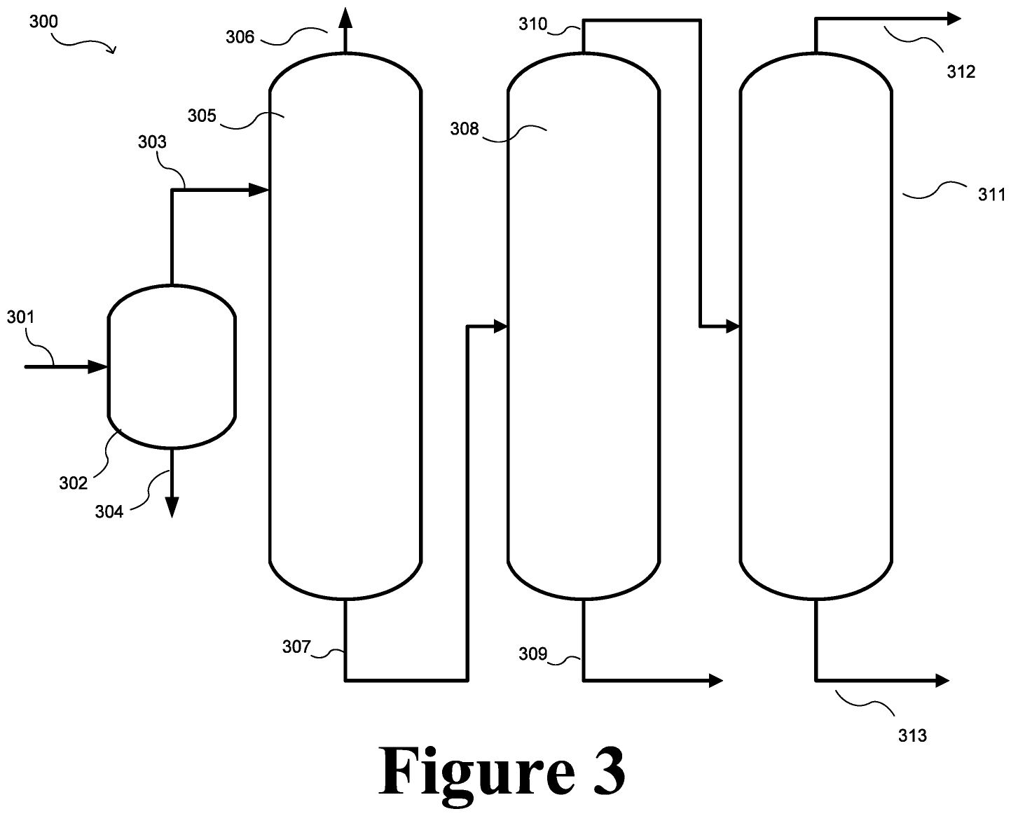

FIG. 3 shows another embodiment of the adiponitrile separation process 300. In this embodiment, an adiponitrile process stream 301 is separated in a flash evaporator 302 to form a first overhead stream 303 and a first bottoms stream 304. The first overhead stream 303 is then separated in a first distillation column 305 to form a lights stream as a second overhead stream 306 and a second bottoms stream 307. The second bottoms stream 307 is then separated in a second distillation column 308 to form a heavies stream as a third bottoms stream 309 and a third overhead, or distillate, stream 310. The third overhead stream 310 is then separated in a third distillation column 311 to form a fourth overhead stream 312 and a TCH stream as a fourth bottoms stream 313.

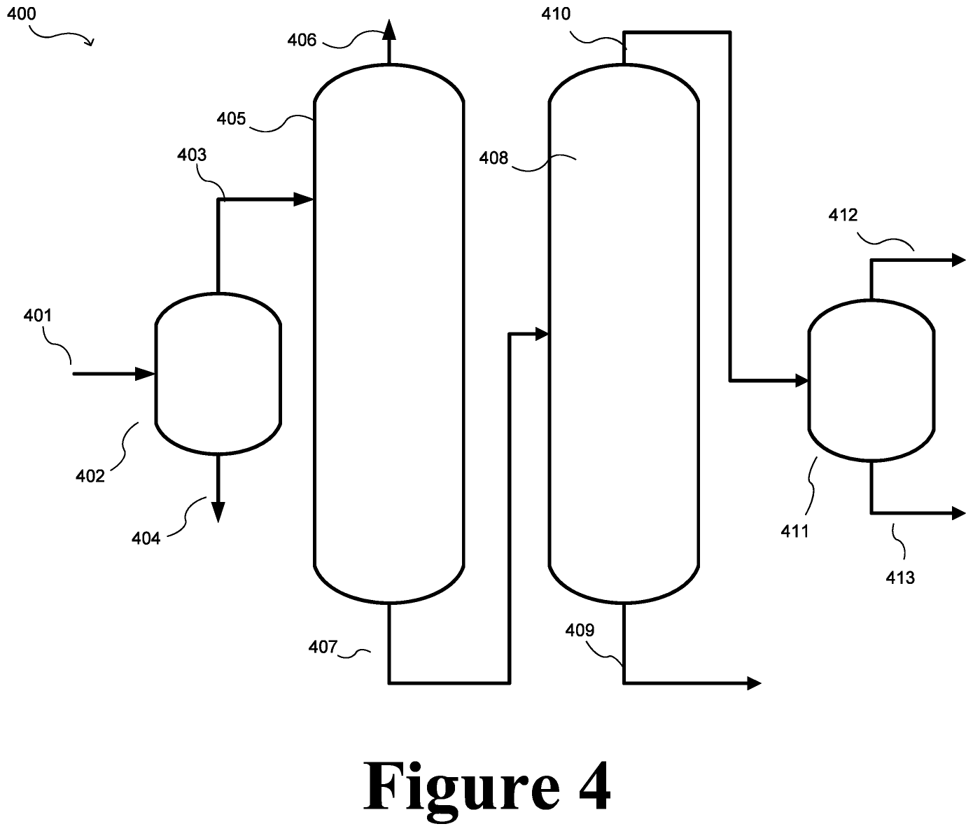

FIG. 4 shows another embodiment of the adiponitrile separation process 400. In this embodiment, an adiponitrile process stream 401 is separated in a flash evaporator 402 to form a first overhead stream 403 and a first bottoms stream 404. The first overhead stream 403 is then separated in a first distillation column 405 to form a lights stream as a second overhead stream 406 and a second bottoms stream 407. The second bottoms stream 407 is then separated in a second distillation column 408 to form a heavies stream as a third bottoms stream 409 and a third overhead, or distillate, stream 410. The third overhead stream 410 is then separated in a flasher 411 to form a fourth overhead stream 412 and a TCH stream as a fourth bottoms stream 413.

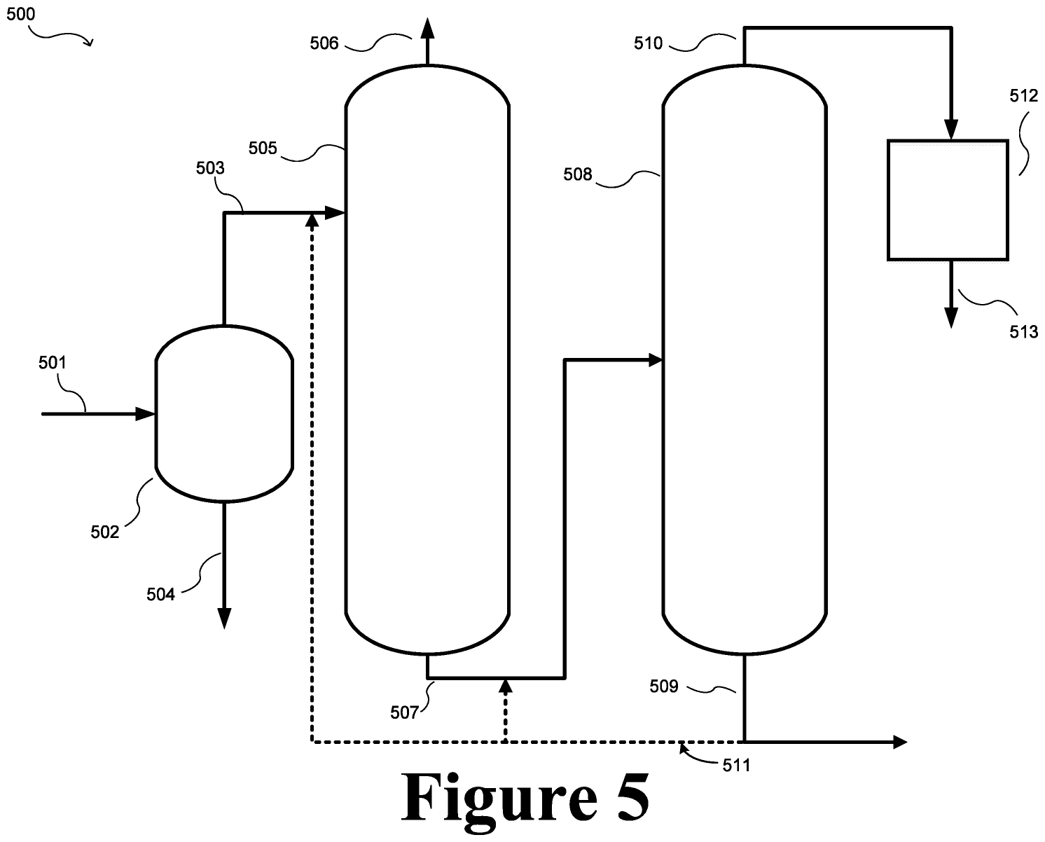

FIG. 5 shows another embodiment of the adiponitrile separation process 500. In this embodiment, an adiponitrile process stream 501 is separated in a flash evaporator 502 to form a first overhead stream 503 and a first bottoms stream 504. The first overhead stream 503 is then separated in a first distillation column 505 to form a lights stream as a second overhead stream 506 and a second bottoms stream 507. The second bottoms stream 507 is then separated in a second distillation column 508 to form a heavies stream as a third bottoms stream 509 and a TCH stream as a third overhead stream 510. This embodiment also features an optional recycle step 511, whereby a portion of the third bottoms stream 509 is recycled to the first overhead stream 503 and/or the second bottoms stream 507. This embodiment also features a treating step 512, whereby the TCH stream 510 is subjected to further treatment to yield a purified TCH stream 513.

The present disclosure will be further understood by reference to the following non-limiting example.

### EXAMPLES

For Examples 1 and 2, an adiponitrile process stream was collected from an adiponitrile production and purification process. The adiponitrile process streams of Examples 1 and 2 were fed to a separation process as described herein, e.g., similar to the separation described in FIG. 1.

The adiponitrile process streams were separated in a wiped film evaporator multiple times times, e.g., two or four times. The multiple passes through the wiped film evaporator produced an overhead (first intermediate adiponitrile stream) and a bottoms (heavies stream), which comprised high-boiling components and solid impurities. The heavies stream was discarded. The compositions of the adiponitrile process stream and the first intermediate adiponitrile stream are provided in Table 1. TCH content, in some cases, included TCH and isomers thereof.

TABLE 1
First Separating Step Flash

| | | First Intermediate | |
|---|---|---|---|
| | Adiponitrile | Adiponitrile Stream | |
| Component | Process Stream | Ex. 1 | Ex. 2 |
| Adiponitrile | 5.0 | 1.0 | 0.7 |
| TCH | 80.0 | 95.0 | 95.9 |
| Lights | 5.0 | 1.5 | 1.8 |
| Heavies | 10.0 | 2.5 | 2.4 |

The first intermediate adiponitrile streams of Examples 1 and/or 2 were distilled in a first distillation column. The first distillation column was operated at a column bottom temperature of about 255° C., and at 1 mmHg and the residence time of the first overhead lights stream in the first distillation column was less than 4 hours. The first distillation column produced an overhead (second intermediate adiponitrile stream), which was beneficially enriched in adiponitrile. Samples of this stream were collected at various times and analyzed. Compositions of these samples are shown in Table 2a. In some cases, the number of cycles in the wiped film evaporator was found to affect the composition of the resulting overhead.

TABLE 2a
Second Separating Step (First Column)

| Component | Second Intermediate Adiponitrile Stream | | |
|---|---|---|---|
| | Sam. 1 | Sam. 2 | Sam. 3 |
| Adiponitrile | 7.1 | 27.09 | 8.93 |
| TCH | 80.3 | 45.77 | 70.49 |
| Lights | 10.6 | 24.59 | 18.97 |
| Heavies | 2.0 | 2.54 | 2.27 |

The first distillation column also produced a second bottoms stream, which contained a high concentration of TCH and some heavies. Samples of this stream were collected at various times and analyzed. Compositions of these samples are shown in Table 2b.

TABLE 2b
Second Separating Step (First Column)

| | Second Bottoms Stream | | | | | | | | | | |
|---|---|---|---|---|---|---|---|---|---|---|---|
| | Sam. | Sam. | Sam. | Sam. | Sam. | Sam. | Sam. | Sam. | Sam. | Sam. | Sam. |
| Comp. | 4 | 5 | 6 | 7 | 8 | 9 | 10 | 11 | 12 | 13 | 14 |
| Adipo | 0.0 | 0.009 | 0 | 0 | 0.003 | 0.006 | 0.004 | 0 | 0 | 0 | 0 |
| TCH | 97.4 | 97.57 | 96.21 | 97.24 | 97.5 | 97.4 | 97.4 | 97.3 | 97.8 | 97.7 | 98.1 |
| Lights | 0.0 | 0.21 | 0.04 | 0.14 | 0.19 | 0.09 | 0.1 | 0.11 | 0.05 | 0.00 | 0.03 |
| Heavies | 2.6 | 2.2 | 3.75 | 2.62 | 2.30 | 2.53 | 2.53 | 2.48 | 2.18 | 2.34 | 1.9 |

The second bottoms streams were then distilled in a second distillation column. The second distillation column was operated at a column bottom temperature of about 263° C., an operating pressure of about 1 mmHg, and the residence time of the second bottoms stream in the second distillation column was less than 4 hours. The second distillation column produced a third bottoms stream (heavies stream). The heavies stream can be recycled and/or discarded. The second distillation column also produced a third overhead stream (TCH stream). Samples of these streams were collected at various times and analyzed. Compositions of these samples are shown in Tables 3a-3d.

TABLE 3a
Second Separating Step (Second Column)

| | TCH Stream | | | | | | | |
|---|---|---|---|---|---|---|---|---|
| Component | Sam. 15 | Sam. 16 | Sam. 17 | Sam. 18 | Sam. 19 | Sam. 20 | Sam. 21 | Sam. 22 |
| Adiponitrile | 0.108 | 0.071 | 0.129 | 0.045 | 0.051 | 0.12 | 0.05 | 0.02 |
| TCH | 98.88 | 98.95 | 98.77 | 97.0 | 97.72 | 98.18 | 99.21 | 99.0 |
| Lights | 0.34 | 0.27 | 0.29 | 0.30 | 0.29 | 0.34 | 0.23 | 0.08 |
| Heavies | 0.67 | 0.67 | 0.81 | 2.61 | 1.89 | 1.34 | 0.51 | 0.89 |

TABLE 3b
Second Separating Step (Second Column)

| | TCH Stream | | | | | | | |
|---|---|---|---|---|---|---|---|---|
| Component | Sam. 22 | Sam. 23 | Sam. 24 | Sam. 25 | Sam. 26 | Sam. 27 | Sam. 28 | Sam. 29 |
| Adiponitrile | 0.046 | 0.026 | 0.021 | 0.016 | 0.03 | 0.02 | 0.038 | 0.023 |
| TCH | 99.12 | 99.06 | 99.03 | 99.65 | 99.03 | 99.28 | 99.34 | 99.48 |
| Lights | 0.46 | 0.14 | 0.08 | 0.11 | 0.22 | 0.16 | 0.12 | 0.19 |
| Heavies | 0.38 | 0.77 | 0.87 | 0.22 | 0.72 | 0.55 | 0.53 | 0.30 |

TABLE 3c
Second Separating Step (Second Column)

| | Heavies Stream | | | | | | | |
|---|---|---|---|---|---|---|---|---|
| Component | Sam. 30 | Sam. 31 | Sam. 32 | Sam. 33 | Sam. 34 | Sam. 35 | Sam. 36 | Sam. 37 |
| Adiponitrile | 0 | 0 | 0 | 0 | 0 | 0 | 0 | 0 |
| TCH | 90.92 | 95.36 | 93.08 | 95.0 | 94.29 | 94.52 | 97.32 | 97.23 |
| Lights | 0.07 | 0.06 | 0.11 | 0.11 | 0.07 | 0.02 | 0.11 | 0.07 |
| Heavies | 9.01 | 4.55 | 6.81 | 4.9 | 5.63 | 5.46 | 2.48 | 2.7 |

TABLE 3d
Second Separating Step (Second Column)

| | Heavies Stream | | | | | | |
|---|---|---|---|---|---|---|---|
| Com- | Sam. | Sam. | Sam. | Sam. | Sam. | Sam. | Sam. |
| ponent | 38 | 39 | 40 | 41 | 42 | 43 | 44 |
| Adipo- | 0 | 0 | 0 | 0 | 0 | 0 | 0 |
| nitrile | | | | | | | |
| TCH | 94.99 | 92.43 | 91.76 | 89.65 | 90.27 | 88.56 | 95.06 |
| Lights | 0.09 | 0 | 0 | 0 | 0 | 0 | 0 |
| Heavies | 4.91 | 7.56 | 8.24 | 10.35 | 9.73 | 11.44 | 4.94 |

As the above Tables show, the separation process carried out in Examples 1 and 2 beneficially produced a (second) intermediate adiponitrile stream in which the adiponitrile concentration was improved over the initial adiponitrile concentration in the feed. Also the process advantageously yielded a highly pure TCH stream. In particular, the purification process resulted in a TCH stream comprising greater than 99 wt. % TCH and comprising no measurable lights (or other impurities). As shown, the concentration of the heavies in the second bottoms stream and/or the heavies stream was maintained within the ranges and limits disclosed herein.

As shown, it was unexpectedly found that as the feed to the column(s) has a higher adiponitrile concentration, the concentration improvement in the column overhead is surprisingly improved. In simulations using similar equipment, when adiponitrile concentration in the column feed was above 10 wt %, then the adipo concentration in the overhead was advantageously higher, e.g., over 50%.

As one benefit, the adiponitrile in the second intermediate adiponitrile stream was employed to form hexamethylene diamine in a separate production process.

### EMBODIMENTS

The following embodiment, among others, are disclosed.

Embodiment 1: A process for producing an intermediate adiponitrile stream, the process comprising: separating an adiponitrile process stream comprising less than 50 wt % adiponitrile, and optionally TCH, to form the intermediate adiponitrile stream comprising at least 5 wt % adiponitrile and a heavies stream comprising high-boiling components and optionally solid impurities; and optionally utilizing at least a portion of the intermediate adiponitrile stream outside of the process.

Embodiment 2: an embodiment of embodiment 1, wherein the separating of the adiponitrile process stream comprises: flashing the adiponitrile process stream to form a first intermediate adiponitrile stream comprising at least 5 wt % adiponitrile and at least 50 wt % TCH and the heavies stream.

Embodiment 3: an embodiment of embodiment 1 or 2, wherein the separating of the adiponitrile process stream comprises: separating the adiponitrile process stream in one or more columns to form a second intermediate adiponitrile stream comprising at least 10 wt % adiponitrile and at least 25 wt % TCH, a heavies stream comprising high-boiling components, and a TCH stream comprising TCH and less than 10 wt. % impurities.

Embodiment 4: an embodiment of any of embodiments 1-3, further comprising purifying the intermediate adiponitrile stream, optionally via one or more distillation columns, to form a purified adiponitrile stream comprising greater than 50 wt % adiponitrile.

Embodiment 5: an embodiment of any of embodiments 1-4, wherein the purified adiponitrile stream comprises greater than 95 wt % adiponitrile and the TCH stream comprises greater than 95 wt % TCH.

Embodiment 6: an embodiment of any of embodiments 1-5, wherein the first intermediate adiponitrile stream comprises less adiponitrile than the second intermediate adiponitrile stream.

Embodiment 7: an embodiment of any of embodiments 1-6, wherein the residence time in the separating step is less than 8 hours.

Embodiment 8: an embodiment of any of embodiments 1-7, wherein the adiponitrile process stream further comprises TCH.

Embodiment 9: an embodiment of any of embodiments 1-8, wherein the utilizing comprises: utilizing adiponitrile in the intermediate adiponitrile stream to form hexamethylene diamine.

Embodiment 10: an embodiment of any of embodiments 1-9, wherein the utilizing comprises: combining the adiponitrile in the intermediate adiponitrile stream form an electrolyte solution.

Embodiment 11: an embodiment of any of embodiments 1-10, wherein the TCH stream comprises: TCH, from 0 wt. % to 0.05 wt. % adiponitrile, from 0 wt. % to 0.1 wt. % di(2-cyanoethyl) amine, from 0 wt. % to 0.05 wt. % cyanovaleramide, and from 0 wt. % to 0.05 wt. % tri(2-cyanoethyl) amine.

Embodiment 12: an embodiment of any of embodiments 1-11, wherein the separating of the adiponitrile process stream comprises: flashing the adiponitrile process stream to form a first intermediate adiponitrile stream comprising at least 5 wt % adiponitrile and at least 50 wt % TCH and the heavies stream, and separating the first intermediate adiponitrile stream in one or more columns to form a second intermediate adiponitrile stream comprising at least 10 wt % adiponitrile, a heavies stream comprising high-boiling components, and a TCH stream comprising at least 25 wt % TCH and less than 10 wt. % impurities.

Embodiment 13: an embodiment of any of embodiments 1-12, wherein the residence time of the intermediate adiponitrile stream in a column of the separating step at temperatures above 230° C. is less than 8 hours.

Embodiment 14: an embodiment of any of embodiments 1-13, wherein the residence time of the intermediate adiponitrile stream in a column of the separating step at pressures above 50 torr is less than 8 hours.

While the invention has been described in detail, modifications within the spirit and scope of the invention will be readily apparent to those of skill in the art. In view of the foregoing discussion, relevant knowledge in the art and references discussed above in connection with the Background and Detailed Description, the disclosures of which are all incorporated herein by reference. In addition, it should be understood that aspects of the invention and portions of various embodiments and various features recited below and/or in the appended claims may be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit.

1. A process for producing an intermediate adiponitrile stream, the process comprising:
separating an adiponitrile process stream comprising less than 50 wt % adiponitrile, and optionally TCH, to form the intermediate adiponitrile stream comprising at least 5 wt % adiponitrile and a heavies stream comprising high-boiling components and optionally solid impurities; and
utilizing at least a portion of the intermediate adiponitrile stream outside of the process.

2. The process of claim 1, wherein the separating of the adiponitrile process stream comprises:
flashing the adiponitrile process stream to form a first intermediate adiponitrile stream comprising at least 5 wt % adiponitrile and at least 50 wt % TCH and the heavies stream.

3. The process of claim 1, wherein the separating of the adiponitrile process stream comprises:
separating the adiponitrile process stream in one or more columns to form a second intermediate adiponitrile stream comprising at least 7 wt % adiponitrile and at least 25 wt % TCH, a heavies stream comprising high-boiling components, and a TCH stream comprising TCH and less than 10 wt. % impurities.

4. The process of claim 1, further comprising purifying the intermediate adiponitrile stream, optionally via one or more distillation columns, to form a purified adiponitrile stream comprising greater than 50 wt % adiponitrile.

5. The process of claim 1, wherein the purified adiponitrile stream comprises greater than 95 wt % adiponitrile and the TCH stream comprises greater than 95 wt % TCH.

6. The process of claim 1, wherein the first intermediate adiponitrile stream comprises less adiponitrile than the second intermediate adiponitrile stream.

7. The process of claim 1, wherein the residence time in the separating step is less than 8 hours.

8. The process of claim 1, wherein the adiponitrile process stream further comprises TCH.

9. The process of claim 1, wherein the utilizing comprises:
utilizing adiponitrile in the intermediate adiponitrile stream to form hexamethylene diamine.

10. The process of claim 1, wherein the utilizing comprises:
combining the adiponitrile in the intermediate adiponitrile stream form an electrolyte solution.

11. The process of claim 1, wherein the TCH stream comprises:
TCH,
from 0 wt. % to 0.05 wt. % adiponitrile,
from 0 wt. % to 0.1 wt. % di(2-cyanoethyl) amine,
from 0 wt. % to 0.05 wt. % cyanovaleramide, and
from 0 wt. % to 0.05 wt. % tri(2-cyanoethyl) amine.

12. The process of claim 1, wherein the separating of the adiponitrile process stream comprises:
flashing the adiponitrile process stream to form a first intermediate adiponitrile stream comprising at least 5 wt % adiponitrile and at least 50 wt % TCH and the heavies stream, and
separating the first intermediate adiponitrile stream in one or more columns to form a second intermediate adiponitrile stream comprising at least 10 wt % adiponitrile, a heavies stream comprising high-boiling components, and a TCH stream comprising at least 25 wt % TCH and less than 10 wt. % impurities.

13. The process of claim 1, wherein the residence time of the intermediate adiponitrile stream in a column of the separating step at temperatures above 230° C. is less than 8 hours.

14. The process of claim 1, wherein the residence time of the intermediate adiponitrile stream in a column of the separating step at pressures above 50 torr is less than 8 hours.
